(19) United States
(12) Patent Application Publication
(10) Pub. No.: US 2001/0005769 A1
(43) Pub. Date: Jun. 28, 2001

(54) A PROCESS FOR THE PREPARATION OF A MIXTURE OF ALKYL PHOENOLS

(76) Inventors: BOLLAPRAGADA S. RAO, PUNE, MAHARASHTRA (IN); KURUNGOT SREEKUMAR, PUNE, MAHARASTRA (IN); TUNDI MADATHIL JYOTHI, PUNE, MAHARASHTA (IN)

Correspondence Address: DICKINSON WRIGHT, 1901 L STREET NW, SUITE 800, WASHINGTON, DC, 20036

(21) Appl. No.: 09/178,576

(22) Filed: Oct. 26, 1998

(51) Int. Cl. C07C 37/00

(52) U.S. Cl. 568/804; 502/300; 502/325; 502/326

(57) ABSTRACT

The present invention relates to a process for the preparation of mixture of alkyl phenols preferably of 2, 6 xylenol and o-cresol using an ortho selective catalyst comprises contacting a catalyst with a feed consisting of an alcohol and a phenolic compound at a temperature ranging between 250 to 400 ° C., at a pressure in the range of ambient to 150 psi and collecting the products at a temperature ranging between −5 to +5° C.

### FIELD OF INVENTION

This is an invention for the preparation of a mixture of alkyl phenols. More particularly it relates to the process of preparation of mixture of 2,6 xylenols and o- cresol using an ortho selective catalyst.

### BACKGROUND OF INVENTION

Alkyl phenols are the compounds having the alkyl groups substituted or replaced either in the phenyl ring or in the OH of the side chain. Cresols, xylenols, alkoxy phenols and trialkyl substituted phenols are some of the important alkyl phenols. These are industrially important chemical intermediates in the manufacture of pharmaceuticals, pesticides, plastics and a variety of chemicals. The stringent specifications and the demand of these chemicals necessitate the development of catalytic systems and the processes for the selective production.

In the prior art the catalysts used for the alkylation of the reactions are ranging from zeolites to metal oxides. U.S. Pat. No. 437174 describes a method for the alkylation of phenols using a Y zeolite but with this both cresols and xylenols are produced and no selectivity for 2,6 Xylenol. Japanese Patent 61-050935 reports the formation of p-alkyl phenols by reaction of phenol with anisole using a zeolite catalyst. US patent 4,283,571 describes a method for the isomerisation of o-cresol to isomeric cresols on zeolite catalyst like ZSM-12. Young in U.S. Pat. No. 4,283,573 reported formation of p-mono alkyl phenols with long chain alcohols and phenols. Many other U.S. patents like 4,197,413 and 4,205,189 describes the formation of alkyl phenols mostly the cresols. The published literature reports the use of catalyst systems varying from alumina, metal oxides, zeolites and acidic catalysts. Most of these reported catalysts are not selective to 2,6 xylenol. So far there is no report in the literature on the use of ferrite systems for the preparation of xylenols. The deactivation rate and the low selectivity to the desired material make these systems less adaptable.

It is therefore desirable to provide a process for the preparation of a mixture of 2,6 xylenol and o-cresol using an ortho selective catalyst composite material which produces negligible amount of cresol and in particular p-cresol, not more than 0.5% other xylenols and removes the drawbacks of the earlier systems. The inventors of the present invention have observed that the use of mixed metal oxide catalyst provided by the present invention removes the above mentioned drawbacks.

The object of the present invention is to provide a process for the production mixture of alkyl phenols more particularly, O-cresol and 2,6-xylenol in high selectivity and with superior activity of the catalyst for longer durations using an ortho alkylation catalyst.

Another object is to provide a process for the said improved catalyst that gives high conversion and high selectivity to 2,6 xylenols with low deactivation rates.

Another object is to provide an improved process for the preparation of an improved catalyst, which need not necessarily be produced in situ.

### SUMMARY OF THE INVENTION

The present invention describes a process for the preparation of a mixture of alkyl phenols preferably of 2,6, xylenol and o-cresol using an ortho selective catalyst belonging to cobalt ferrite type which gives 2,6 selectivity to at least 60% and the phenol conversion to at least 75% with negligible amount of other cresols in particular p-cresol.

### DETAILED DESCRIPTION OF THE INVENTION

Accordingly the present invention provides a process for the preparation of mixture of alkyl phenols, which comprises contacting a catalyst with a feed consisting of an alcohol and a phenolic compound at a temperature ranging between 250 to 400° C., at a pressure in the range of ambient to 150 psi, collecting the products at a temperature ranging between −5 to +5° C.

In another embodiment the alcohol used is such as be lower alcohols such as methanol, or any other methylating agent such as dimethyl carbonate.

In yet another embodiment the phenolic compounds used is such as be phenol, methoxy phenols, o-cresol or methyl methoxy phenol.

In another embodiment the ratio of alcohol to phenolic compound is such as vary from 7:1 (W/W).

In still another embodiment the space velocity of the reaction mixture is such as be in the range of 1 to 5 WHSV (Weight hourly space velocity expressed in terms of gms of feed per gram of catalyst per hour.

In another embodiment of the present invention the catalyst used has general formula

M_{A}N_{B}Fe₂O₄

Wherein M is a divalent metal such as cobalt, nickel, zinc, chromium, magnesium, mangenese and cadmium.

N is any divalent metal other than M,

A=0 to 1 and B=0 to 1 and A+B=1

Characterized by the XRD pattern as given in the table-1

TABLE 1

| d A° | I/Io | hkl |
|---|---|---|
| 4.89 | 8 | 111 |
| 2.95 | 40 | 220 |
| 2.51 | 100 | 311 |
| 2.41 | 9 | 222 |
| 2.08 | 19 | 400 |
| 1.70 | 8 | 422 |
| 1.61 | 45 | 333, 511 |
| 1.48 | 65 | 440 |
| 1.32 | 3 | 620 |
| 1.28 | 13 | 533 |
| 1.20 | 3 | |
| 1.12 | 5 | |
| 1.09 | 20 | |
| 1.05 | 4 | |
| 0.99 | 2 | |
| 0.97 | 8 | |

having a surface area of 200 to 400 sq. Mts./gm and a pore size varying from 10 to 50 Å

In still another embodiment the catalyst used is CoFe₂O₄ which is seletced from the group M₁M₂M₃O₄ where M₁ is cobalt M₂ is nickel and M₁ =1.0, 0.2 and 0.5 and M₂ is 0, 0.5 and 0.8 and M₃ is Fe wherein the selectivity of xylenol is not less than 60% and conversion of phenol is not less than 75%

In a feature of the present invention the catalyst is prepared as per the procedure which comprises preparing the solutions of the source of iron, a source of a divalent metal and optionally a source of another divalent metal other than cobalt, separating the undissolved solids from the solutions by conventional methods, mixing the solutions in the stiotiometric ratio, adding this reaction mixture to an alkali solution, further adjusting the pH of the solution in the range of 9.0 to 10, heating the mixture to a temperature ranging between 50 to 70° C., digesting the mixture at this temperature for a period ranging between 1 to 2 hrs. to obtain the precipitate of the product, cooling the precipitate, washing with water to obtain the product, drying the product at temperature ranging between 80 to 100° C. for a period of 24 to 48 hrs., calcining the product at a temperature ranging between 300 to 500° C. for 10 to 48 hrs.

The process of the present invention is described herein below with reference to following examples which are illustrative only and should not be construed to limit the scope of the present invention in any manner.

Examples 1 to 5 describe preparation of various catalyst material having different compositions.

### EXAMPLE-1

124 gms. of cobalt nitrate, and 344.2 gms. of ferric nitrate were dissolved separately each in 500 ml. of deionised water, and filtered to remove any suspended particles. These solutions were mixed together and an alkali solution containing 80 gms of liquor ammonia dissolved in 200 ml. of water was added to the mixture dropwise. The pH of the resultant mixture was adjusted to 9.4 by adding dilute ammonia solution. The mixture was heated to 70° C. and digested for 2 hrs. The precipitated material was cooled to room temperature, filtered and washed free of nitrate ions. The precipitate was dried at 100° C. for 36 hrs. and calcined at 300° C. for twelve hrs. in flowing air. The dried material was powdered and sieved to the size of ˜5 to 50 mesh. This gives catalyst CoFe₂O₄ (100 gms)

### EXAMPLE-2

130 gms. of nickel nitrate, and 344.2 gms. of ferric nitrate were dissolved separately each in 500 ml. of deionised water, and filtered to remove any suspended particles. These solutions were mixed together and an alkali solution containing 80 gms of liquor ammonia dissolved in 200 ml. of water was added to the mixture dropwise. The pH of the resultant mixture was adjusted to 9.4 by adding dilute ammonia solution. The mixture was heated to 70° C. and digested for 2 hrs. The precipitated material was cooled to room temperature, filtered and washed free of nitrate ions. The precipitate was dried at 100° C. for 36 hrs. and calcined at 350° C. for twelve hrs. in flowing air. The dried material was powdered and sieved to the size of ˜5 to 50 mesh. This gives catalyst of NiFe₂O₄ (100 gms)

### EXAMPLE-3

This example illustrates the preparation of mixed cobalt nickel oxide ferrite catalyst. 62 gms. of cobalt nitrate, 65 gms. Of Nickel nitrate and 344.2 gms. of ferric nitrate were dissolved separately each in 500 ml. of deionised water, and filtered to remove any suspended particles. These solutions were mixed together and an alkali solution containing 80 gms of liquor ammonia dissolved in 200 ml. of water was added to the mixture dropwise. The pH of the resultant mixture was adjusted to 9.9 by adding dilute ammonia solution. The mixture was heated to 60° C. and digested for 2 hrs. The precipitated material was cooled to room temperature, filtered and washed free of nitrate ions. The precipitate was dried at 110° C. for 48 hrs. and calcined at 380° C. for twelve hrs. in flowing air. The dried material was powdered and sieved to the size of ˜5 to 50 mesh. This gives Co_{(0.5)}Ni_{(0.5)}Fe₂O₄ catalyst (100 gms.)

### EXAMPLE-4

This example illustrates the preparation of mixed cobalt nickel oxide ferrite catalyst. 25.0 gms. of cobalt nitrate, 104 gms. of nickel nitrate and 344.2 gms. of ferric nitrate were dissolved separately each in 500 ml. of deionised water, and filtered to remove any suspended particles. These solutions were mixed together and an alkali solution containing 80 gms of liquor ammonia dissolved in 200 ml. of water was added to the mixture dropwise. The pH of the resultant mixture was adjusted to 9.9 by adding dilute ammonia solution. The mixture was heated to 60° C. and digested for 2 hrs. The precipitated material was cooled to room temperature, filtered and washed free of nitrate ions. The precipitate was dried at I 110° C. for 48 hrs. and calcined at 300° C. for twelve hrs. in flowing air. The dried material was powdered and sieved to the size of ˜5 to 50 mesh. This gives Co_{(0.2)}Ni_{(0.8)}Fe₂O₄ catalyst (100 gms.)

### EXAMPLE-5

This example illustrates the preparation of mixed cobalt nickel oxide ferrite catalyst. 100 gms. of cobalt nitrate, 26 gms. Of Nickel nitrate and 344.2 gms. of ferric nitrate were dissolved separately each in 500 ml. of deionised water, and filtered to remove any suspended particles. These solutions were mixed together and an alkali solution containing 80 gms of liquor ammonia dissolved in 200 ml. of water was added to the mixture dropwise. The pH of the resultant mixture was adjusted to 9.9 by adding dilute ammonia solution. The mixture was heated to 60° C. and digested for 2 hrs. The precipitated material was cooled to room temperature, filtered and washed free of nitrate ions. The precipitate was dried at 110° C. for 48 hrs. and calcined at 500° C. for twelve hrs. in flowing air. The dried material was powdered and sieved to the size of ˜5 to 50 mesh. This gives Co_{(0.8)}Ni_{(0.2)}Fe₂O₄ catalyst (100 gms.)

Examples 6 to 13 describe the process for the preparation of mixtures of alkyl phenols. Example 14 describe the process with various catalysts prepared as per examples 2 to 5. Example 15 describes the use of dilution of feed and its effect on product selectivity. In examples 6 to 13 the catalyst used is CoFe₂O₄

### EXAMPLE-6

10 gms. of the catalyst material is pressed and crushed to particles of 10-20 mesh and placed at the center of a reactor maintained at a constant temperature of 350° C. The feed containing methanol and phenol at a ratio of 4-1 (mol/mol) as fed in to the catalyst system at a space velocity of 1. The outlet of the reactor was cooled by chilled water and the products analyzed on a GC. column. The results are shown in table-2

TABLE 2
Feed WHSV 1 Mole ratio Pheno:MeOH = 1:4

| Product distribution (wt %) | |
|---|---|
| Non Aromatics | NIL |
| Benzene | 0.12 |
| Toluene | 0.66 |
| Anisole | 1.76 |
| O-Cresol | 23.97 |
| 2,6 Xylenol | 73.19 |
| Other alkylates | 0.18 |
| Others | 0.12 |
| Phenol conversion | 80.78 |
| O-Cresol selectivity | 28.0 |
| 2,6 Xylenol selectivity | 72.0 |

### EXAMPLE-7

The feed (methanol and phenol) at a mole ratio varying between 1 and 7 was contacted over the 10 grams of catalyst at a space velocity ranging between 1 and 5. The temperature of the catalyst bed was maintained at 350° C., The outlet of the reactor was cooled by chilled water and the products analyzed on a GC. column. The results are shown in table-3.

WHSV 1 h⁻¹, reaction temperature 350° C.

TABLE 3

| Ratio (Me/Ph) | 2 | 5 | 7 |
|---|---|---|---|
| Product distribution, Wt % | | | |
| Non Aromatics | Nil | Nil | 0.43 |
| Benzene | 0.14 | 0.10 | 0.03 |
| Toluene | 0.70 | 0.45 | 0.25 |
| Anisole | 3.90 | 3.70 | 3.11 |
| o-Cresol | 23.10 | 21.31 | 18.30 |
| 2,6 Xylenol | 63.40 | 74.40 | 76.14 |
| Other Alkyl Phenols | 5.15 | 0.04 | 0.87 |
| Others | 3.61 | Nil | 0.87 |
| Phenol Conversion | 48.63 | 81.77 | 92.25 |
| o-Cresol Selectivity | 23.13 | 22.27 | 18.62 |
| 2,6 Xylenol Selectivity | 63.35 | 77.72 | 77.40 |

### EXAMPLE-8

This example shows the effect of space velocity on the product distribution and the activity of the catalyst. In order to investigate this a set of experiments with the feed weight varied from 1-5 gm. per gm. of catalyst. The products are collected after cooling and analyzed f6r calculating the yields and selectivities . Table-4 includes the data of this study.

TABLE 4
Reaction temperature 350° C., Ph to Me mole ratio 1:7

| WHSV | 1.5 | 2.5 | 4 |
|---|---|---|---|
| Product Distribution, Wt % | | | |
| Non Aromatics | 0.43 | 0.44 | 0.57 |
| Benzene | 0.03 | 0.13 | 0.19 |
| Toluene | 0.25 | 0.13 | 0.19 |
| Anisole | 3.11 | 8.44 | 9.97 |
| o-Cresol | 18.30 | 41.35 | 48.22 |
| 2,6 Xylenol | 76.14 | 47.76 | 38.52 |
| Other Alkyl Phenols | 0.87 | 0.94 | 1.20 |
| Others | 0.87 | 0.80 | 1.15 |
| Phenol Conversion | 92.25 | 78.72 | 52.70 |
| o-Cresol Selectivity | 18.62 | 42.08 | 49.37 |
| 2,6 Xylenol Selectivity | 77.48 | 48.61 | 39.44 |

### EXAMPLE-9

This example shows the effect of temperature on selectivity of the product formation. The feed of phenol and methanol in the ratio of 1:4 was fed to a bed of catalyst maintained at temperatures ranging between 300-400° C. The products were collected at the outlet of the reactor and analyzed for the composition. The results are tabulated in table-5

TABLE 5
WHSV 1 h⁻¹, Ph to Me mole ratio 1:7

| Temperature, ° C. | 300 | 325 | 350 | 400 |
|---|---|---|---|---|
| Product Distribution wt % | | | | |
| Non Aromatics | Nil | 0.35 | 0.43 | 0.47 |
| Benzene | Nil | 1.65 | 0.03 | 2.57 |
| Toluene | Nil | Nil | 0.25 | 4.45 |
| Anisole | 1.76 | 3.23 | 3.11 | 0.75 |
| o-Cresol | 64.42 | 44.63 | 18.3 | 40.32 |
| 2,6 Xylenol | 33.73 | 49.56 | 76.14 | 48.98 |
| Other Alkyl Phenols | Nil | 0.23 | 0.87 | 1.23 |
| Others | 0.08 | 0.35 | 0.87 | 1.23 |
| Phenol Conversion | 60.00 | 85.69 | 92.25 | 95.77 |
| o-Cresol Selectivity | 63.45 | 44.63 | 18.62 | 40.18 |
| 2,6 Xylenol Selectivity | 33.17 | 49.56 | 77.48 | 48.80 |

### EXAMPLE-10

A feed consisting of ortho cresol and methanol in the mole ratio 4 was contacted with the catalyst material at a temperature of 350° C. and the products were collected and analyzed. The results are tabulated in table-6

TABLE 6
WHSV 2 h⁻¹, reaction temperature 350° C. Me to o-Cresol = 4

| Product Distribution (Wt %) | |
|---|---|
| Non Aromatics | 0.06 |
| Benzene | 0.07 |
| Toluene | 0.05 |
| Anisole | 1.49 |
| Phenol | 3.04 |
| 2,6 Xylenol | 94.37 |
| Methyl Anisole | 0.36 |
| Other Alkyl Phenols | 0.20 |
| Others | 0.36 |
| o-Cresol Conversion | 82.75 |
| 2,6 Xylenol Selectivity | 94.44 |

### EXAMPLE-11

In this example the feed consisting of anisole and methanol in the mole ratio 1:4 was passed over the catalyst bed at 350° C. The products were collected and analyzed. The results are given in table-7

TABLE 7
WHSV = 1, Temp 350° C. Anisole: Methanol 1:4

| Product Distribution Wt % | |
|---|---|
| Non aromatics | NIL |
| Benzene | NIL |
| Toluene | NIL |
| Phenol | 28.32 |
| O-cresol | 33.03 |
| 2,6 Xylenol | 35.09 |
| Other alkylates | 2.41 |
| Others | 1.15 |
| Conversion anisole % | 87.2 |
| Selectivity O-cresol | 45.2 |
| Selectivity 2,6 Xylenol | 50.2 |

### EXAMPLE-12

The catalyst composite material of example 1 was contacted with a feed consisting of dimethylcarbonate and phenol in the mole ratio 2 and 3 at a temperature of 350° C., WHSV=1. The product analysis is given in table-8

TABLE 8
WHSV = 1, Temp 350° C., WHSV 1 h⁻¹, Reaction temperature 300° C.

| DMC to Ph mole ratio | 2 | 3 |
|---|---|---|
| Product Distribution, Wt % | | |
| Non aromatics | 3.00 | 4.18 |
| Anisole | 9.28 | 6.19 |
| Methyl anisole | Nil | Nil |
| o-Cresol | 62.33 | 64.08 |
| 2,6 Xylenol | 24.78 | 25.47 |
| Others | 0.61 | 0.08 |
| Phenol conversion | 42.33 | 60.08 |
| o-Cresol selectivity | 62.3 | 64.08 |
| 2,6 Xylenol selectivity | 24.78 | 25.47 |

### EXAMPLE-13

Catalyst of general formula described hereinbefore (M_{A}N_{B}Fe₂O₄) were used for the preparation of the alkyl phenolic mixture. The reaction conditions are described hereinbelow.

WHSV 1 h⁻¹, Ph to Me mole ratio 7, reaction temperature 350° C.

TABLE 9

| Catalyst | | | | | |
|---|---|---|---|---|---|
| Composition | M1M3₂O₄ | M1_{0.8}M2_{0.2}M3₂O₄ | M1_{0.5}M2_{0.5}M3₂O₄ | M1_{0.2}M2_{0.8}M3₂O4 | M2M3₂O₄ |
| Product Distribution (wt %) | | | | | |
| Non Aromatics | 0.43 | 0.12 | 0.48 | 0.12 | 0.88 |
| Benzene | 0.03 | 0.05 | Nil | 0.24 | 0.69 |
| Toluene | 0.25 | 0.04 | Nil | 0.03 | 1.06 |
| Anisole | 3.11 | 3.56 | 0.74 | 0.06 | Nil |
| o-Cresol | 18.30 | 47.11 | 32.14 | 28.33 | 70.98 |
| 2,6 Xylenol | 76.14 | 48.16 | 65.03 | 70.81 | 18.07 |
| Other Alkyl Phenols | 0.87 | 0.49 | 0.54 | 0.16 | 4.67 |
| Others | 0.87 | 0.47 | 1.07 | 0.25 | 3.63 |
| PhOH Conversion | 92.25 | 44.72 | 79.32 | 88.06 | 43.25 |
| o-Cresol Selectivity | 18.62 | 47.10 | 32.70 | 28.27 | 70.97 |
| 2,6 Xylenol sel. | 77.48 | 48.16 | 65.95 | 70.65 | 18.07 |

### EXAMPLE-14

This example illustrates the effect of dilution of the feed using water. A feed consisting of a mixture of phenol, methanol and water is contacted on a catalyst composite material. The results are presented in table-12.

WHSV 1 h⁻¹ reaction temperature 350° C., flow rate 4 ml/hr

TABLE 10

| | Without | 1:1:6 | 1:2:5 |
|---|---|---|---|
| Feed composition | Water | (Ph:H₂O:Me) | (Ph:H₂O:Me) |
| Product Distribution (wt %) | | | |
| Non Aromatics | 0.43 | Nil | Nil |
| Benzene | 0.03 | 0.72 | 0.40 |
| Toluene | 0.25 | 0.70 | 0.48 |
| Anisole | 3.11 | 0.11 | 0.16 |
| o-Cresol | 18.30 | 41.74 | 29.65 |
| 2,6 Xylenol | 76.14 | 76.00 | 68.50 |
| Other Alkyl Phenols | 0.87 | 0.50 | 0.54 |
| Others | 0.87 | 0.23 | 0.27 |
| PhOH Conversion | 92.25 | 89.96 | 62.18 |
| o-Cresol Selectivity | 18.62 | 21.73 | 29,63 |
| 2,6 Xylenol Selectivity | 77.48 | 75.98 | 68.50 |

We claim:

1. An improved process for the preparation of mixture of alkyl phenols, which comprises contacting a catalyst with a feed consisting of an alcohol and a phenolic compound at a temperature ranging between 250 to 400° C., at a pressure in the range of ambient to 150 psi, collecting the products at a temperature ranging between −5 to +5° C.

2. A process as claimed in claim 1, wherein the alcohol used is from lower alcohols such as methanol, or other methylating agents such as dimethyl carbonate.

3. A process as claimed in claim 1, wherein the phenolic compounds used is such as phenol, methoxy phenols, o-cresol or methyl methoxy phenol.

4. A process as claimed in claim 1, wherein the ratio of to phenolic compound to alcohol varies from 1:1 to 1:7 (mol/mol).

5. A process as claimed in claim 1 wherein the catalyst used has general formula
M_{A}N_{B}Fe₂O₄
Wherein M is a divalent metal such as cobalt, nickel, zinc, chromium, manganese, magnesium and cadmium.
N is any divalent metal other than M,
A=0 to 1 and B=0 to 1 and A+B=1
Characterized by the XRD pattern as given in the table-1, having a surface area of 300 to 400 sq. Mts./gm and a pore size varying from 10 to 50 Å

6. A process as claimed in claim 1 wherein the catalyst used is CoFe₂O₄ which is seletced form the group consisting of M₁M₂M₃O₄ where M₁ is cobalt M₂ is nickel and M₁=1.0, 0.2 and 0.5 and M₂ is 0, 0.5 and 0.8 and M₃ is Fe.

7. A process as claimed in claims 1 to 6 wherein the catalyst used results in achieving the selectivity of 2,6 xylenol to at least 60% and conversion of phenol to at least 75%

8. A process as claimed in claim 1, wherein the space velocity of the reaction mixture is in the range of 1 to 5 WHSV (Weight hourly space velocity expressed in terms of gms of feed per gram of catalyst per hour).
